SEQUENCE LISTING (8 sequences)

SEQ ID NO: 1 - protein, 34 aa, Artificial Sequence
  Synthetic Construct
RLPAGWEQRMDVKGRPYFVDHVTKSTTWEDPRPE

SEQ ID NO: 2 - protein, 34 aa, Unknown
  Natural WW domain also called Nedd4.3 Kanelis, V., Rotin D. & Forman-Kay, JD (2001) Solution structure of a Nedd4 WW domain-ENaC peptide complex, Nat Struct Biol 8 407-12.
PLPPGWEERTHTDGRVFFINHNIKKTQWEDPRMQ

SEQ ID NO: 3 - protein, 13 aa, Unknown
  Group I proline-rich peptides
EYPPYPPPPYPSG

SEQ ID NO: 4 - protein, 45 aa, Artificial Sequence
  Synthetic Construct
AGAGAGPEGAGAGAGPEGRGDSAGPEGAGAGAGPEGAGAGAGPEG

SEQ ID NO: 5 - protein, 33 aa, Artificial Sequence
  Polyhistidine-tag
MGSSHHHHHHSSGLVPRGSSSGHIDDDDKVDGT

SEQ ID NO: 6 - protein, 253 aa, Artificial Sequence
  Synthetic Construct
  REPEAT (125)..(215): Repeated between 1-5 times.
MGSSHHHHHHSSGLVPRGSSSGHIDDDDKVDGTRLPAGWEQRMDVKGRPYFVDHVTKSTTWEDPRPEGTLDELAGAGAGPEGAGAGAGPEGRGDSAGPEGAGAGAGPEGAGAGAGPEGELLDGTRLPAGWEQRMDVKGRPYFVDHVTKSTTWEDPRPEGTLDELAGAGAGPEGAGAGAGPEGRGDSAGPEGAGAGAGPEGAGAGAGPEGELLDGTRLPAGWEQRMDVKGRPYFVDHVTKSTTWEDPRPEGTLE

SEQ ID NO: 7 - protein, 253 aa, Artificial Sequence
  Synthetic Construct
  REPEAT (125)..(214): Repeated from 1 to 5 times
MGSSHHHHHHSSGLVPRGSSSGHIDDDDKVDGTPLPPGWEERTHTDGRVFFINHNIKKTQWEDPRMQGTLDELAGAGAGPEGAGAGAGPEGRGDSAGPEGAGAGAGPEGAGAGAGPEGELLDGTPLPPGWEERTHTDGRVFFINHNIKKTQEDPRMQGTLDELAGAGAGPEGAGAGAGPEGRGDSAGPEGAGAGAGPEGAGAGAGPEGELLDGTPLPPGWEERTHTDGRVFFINHNIKKTQWEDPPRMQGTLE

SEQ ID NO: 8 - protein, 18 aa, Artificial Sequence
  Synthetic Construct
AGAGAGPEGAGAGAGPEG

(19) United States
(12) Patent Application Publication
(10) Pub. No.: US 2025/0295584 A1
(43) Pub. Date: Sep. 25, 2025

(54) Dynamically crosslinked injectable hydrogels with chemically stabilized multilamellar vesicles

(71) Applicant: The Board of Trustees of the Leland Stanford Junior University, Stanford, CA (US)

(72) Inventors: Sarah C. Heilshorn, Mountain View, CA (US); Michael J. Kratochvil, San Mateo, CA (US)

(21) Appl. No.: 17/928,058

(22) PCT Filed: Jun. 7, 2021
(86) PCT No.: PCT/US2021/036095

§ 371 Date: Nov. 28, 2022

Related U.S. Application Data
(60) Provisional application No. 63/035,510, filed on Jun. 5, 2020.

(51) Int. Cl. A61K 9/06; A61K 9/00; A61K 9/127; A61K 47/24

(52) U.S. Cl. CPC A61K 9/06; A61K 9/0019; A61K 9/127; A61K 47/24

(57) ABSTRACT

An injectable hydrogel for drug delivery is provided having a first component of repeating protein domains, and a second component cross-linked with the first component. The protein domain in the repeating protein domains is a WW protein sequence interspersed with hydrophilic protein sequences (repeats ranges from 2 to 10). The second component is an inter-bilayer crosslinked multilamellar vesicle. The vesicle is capable of containing drug cargo and eluting the drug cargo. The vesicle is derived from a first lipid and a second lipid. The surface of the vesicle has tethers, which are cross-linked with protein domains of the first component. Control over elution rate of drug cargo is accomplished through stabilization of the vesicles. Gelation occurs in a simple and rapid mixing of the two components without the necessity of additional crosslinking stimuli. Tuning of elution rate is achieved while not losing injectability. Bioresorbable components are used.

### SEQUENCE LISTING

This application includes a sequence listing submitted in written form and in computer readable form.

### FIELD OF THE INVENTION

This invention relates to injectable hydrogels for drug delivery.

### BACKGROUND OF THE INVENTION

Currently, there are no easy ways to locally deliver a drug and control the release of that drug over time. Injectable hydrogel systems can serve as a biocompatible solution to these drug delivery issues. Hydrogels are water-swollen polymer matrices that are held together through crosslinking of the polymer components into an expansive network. Physically cross-linked hydrogels feature dynamic breaking and reforming of cross-links that permit the delivery of the formed hydrogel via syringe injection. This class of material is well suited for biological delivery of drugs, but most payloads encapsulated within conventional hydrogel systems rapidly diffuse out of the material. This invention addresses this issue by designing controlled drug releasing, physically-crosslinked hydrogels that have chemically stabilized and tunable drug-loaded vesicles as a component of the hydrogel, and which are injectable hydrogels.

### SUMMARY OF THE INVENTION

The present invention provides an injectable hydrogel for drug delivery is provided having a first component of repeating protein domains, and a second component cross-linked with the first component. The protein domain in the repeating protein domains is a WW protein sequence interspersed with hydrophilic protein sequences (repeats ranges from 2 to 10). The WW protein sequence is selected from SEQ ID 1, SEQ ID 2, SEQ ID 3, SEQ ID 4, SEQ ID 5, SEQ ID 6, or SEQ ID 7. AGAGAGPEGAGAGAGPEG (SEQ ID: 8) is a hydrophilic spacer between the first and second components.

The second component is an inter-bilayer crosslinked multilamellar vesicle. The vesicle is capable of containing drug cargo and eluting the drug cargo. The vesicle is derived from a first lipid (e.g. DOPC) and a second lipid (e.g. MPB). The surface of the vesicle has tethers, which are cross-linked with protein domains of the first component. An example of the tethers is proline peptide tethers.

### Definitions or Design Aspects

Multilamellar vesicles are spherical liposomes with several (greater than one) lamellar phase lipid bilayers.
A first lipid as used in this invention can be any phospholipid that is net neutral (including zwitterionic lipids). The first lipid can also be a mixture of net neutral lipids.
The second lipid as used in this invention must contain a reactive group that does not form covalent bonds with the first lipid, and retains reactivity through the multilamellar vesicle fabrication process.
Tethers must undergo reversible interactions in the gel network formation process to allow injectability when shear stress is applied. The tether would need to have a thiol as an end group to facilitate the functionalization of the tether onto the surface of the vesicles via the maleimide on the MPBs.

Embodiments of the invention have at least one of the following advantages:
Provide control over elution rate of drug cargo through stabilization of the vesicles, rather than changing of the mechanical properties of the hydrogel.
Gelation occurs in a simple and rapid mixing of the two components without the necessity of additional crosslinking stimuli (i.e. no temperature change, pH change, irradiation, or any other crosslinking stimulus needed).
Allow for tuning of elution rate while not losing injectability.
Use bioresorbable components (e.g. phospholipids, proteins).

### BRIEF DESCRIPTION OF THE DRAWINGS

FIGS. 1-2 show according to exemplary embodiments of the invention a comparison of the so-called MITCH (Mixing-Induced Two-Component Hydrogel) system of U.S. Pat. No. 9,011,914 (FIG. 1) and the hydrogel drug delivery system or material of this invention (FIG. 2). 110 or 210=Proline Rich Peptide (P-peptide) and 120 or 220=WW domain sequence. The diamonds are the locations of the cell adhesive domain sequences that intersperse the WW domain sequences in the C7 protein. The diamonds can also be referred to as cell compatibility sequences.

FIG. 3 shows the formation of the injectable hydrogel for drug delivery according to exemplary embodiments of the invention by combining the first (e.g. C7) and second component (DEN—Drug-Eluting Nanodroplet).

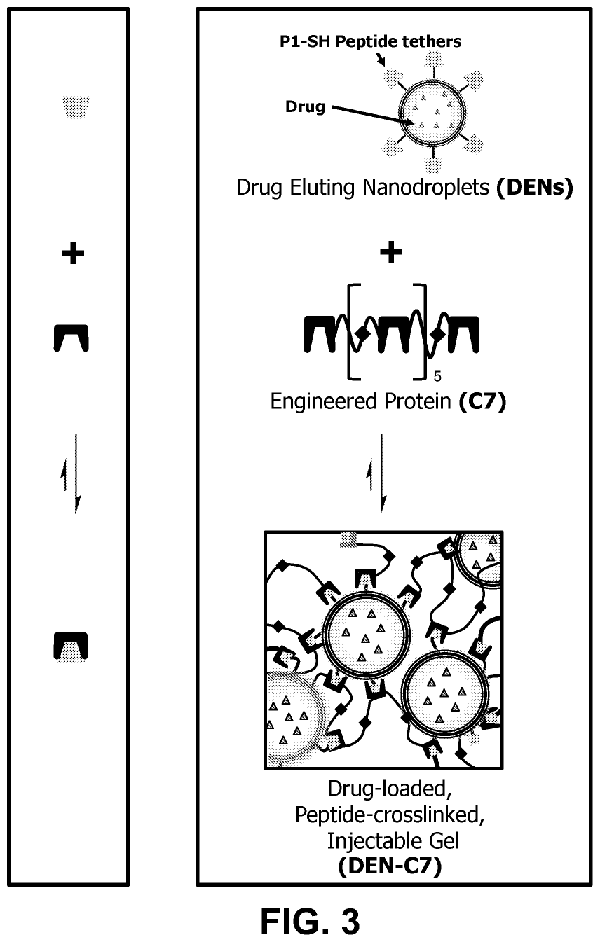

FIG. 4 shows according to exemplary embodiments of the invention a flow diagram of the formation of C7-DEN.

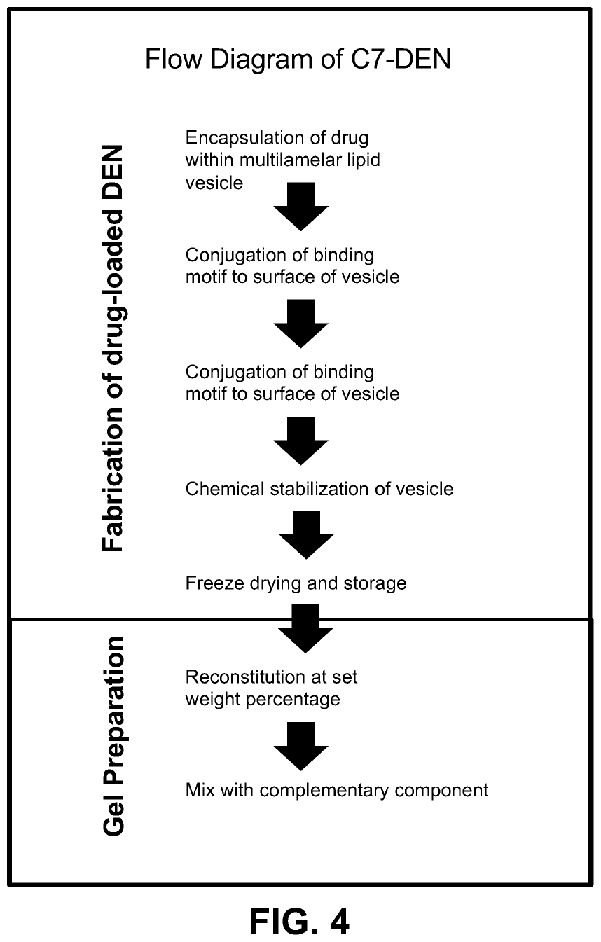

### DETAILED DESCRIPTION

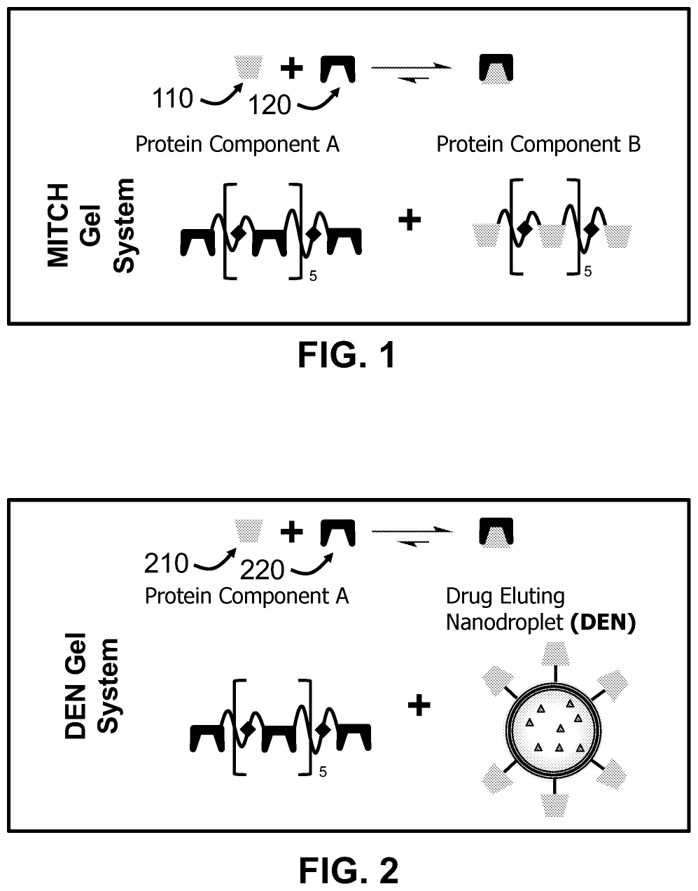

The basis of this invention is the formation of gels through the mixing of two complementary components to form an injectable hydrogel with tunable drug release. Previously, as taught in U.S. Pat. No. 9,011,914, Applicant of this invention provided the design of a Mixing-Induced Two-Component Hydrogel (MITCH) system, that formed physically-crosslinked hydrogels through the mixing of two peptide components together at physiological conditions without the need of external stimulus (e.g. chemical crosslinkers, photo-illumination, change in temperature or pH, etc.) (FIG. 1).

In this invention, a stabilized multilamellar Drug-Eluting Nanodroplet (termed DEN(s)) is used as a drug depot and hydrogel component (FIG. 2). This drug delivery hydrogel system, composed of DENs and the C7 protein from the MITCH system, is a tunable extended release of payloads from the vesicles within a biocompatible hydrogel material that is deliverable via injection by either syringe or catheters.

The C7-DEN hydrogel system is a linear engineered protein (termed C7) and an inter-bilayer crosslinked multilamellar vesicle that serves as a drug-eluting nanodroplet surface-functionalized with the proline rich peptides (termed P1). The C7 protein is an engineered synthetic protein that contains seven repeats of the computationally designed WW domain interspersed with a hydrophilic, random coil peptide sequence. The DENs are composed of a mixture of two lipids:
1,2-dioleoyl-sn-glycero-3-phosphocholine (DOPC), and
1,2-dioleoyl-sn-glycero-3-phosphoethanolamine-N-[4-(p-maleimidophenyl) butyramide] (MPB).

Following hydration and agitation of dried MPB and, multilamellar vesicles are formed. The MPB component features a thiol-reactive maleimide group, which is used to chemically stabilize the bilayers through the treatment with a dithiol small molecule. Tuning the proportion of dithiol to MPB provides control over the relative stability of the resulting DENs. Residual MPB on the surface of the DENs are then functionalized with P1-thiols, so as to present Pls on the surface of the vesicle. The mixing of C7 with P1-functionalized DENs leads to sol-gel phase transition at physiological conditions through the heterodimerization among C and P domains on the C7 and DEN components, respectively. The resultant hydrogel is shear thinning, due to the reversible nature of the C-P physical crosslinks, and thus can be injected through a syringe needle.

Previously, the MITCH-PEG system, composed of C7 and a P1-functionalized multi-arm polyethylene glycol (PEG) has been demonstrated to provide significant protection to encapsulated cells from disruptive mechanical forces that occur during injection as compared to saline alone. Key aspects of the multi-arm PEG in the system are:
It is readily water soluble,
Is multi-armed and thus can have multiple (>2) interactions leading to crosslinking and gelations, and
Is functionalized at the end of each of the arms to facilitate the crosslinking interactions.

Like the MITCH-PEG system, the C7-DEN material has the same physical properties to provide protection to embedded cells or liposomal particles during injection.

Key aspects of the embodiments of this invention are, for example:
Unique use of drug releasing depot as the crosslinking center for hydrogel formation.
Rapid and reversible crosslinking that eliminates the need for curing times of the hydrogel, and that can be mixed and delivered by injection.
Encapsulation of drug within chemically-stabilized multilamellar vesicle adds control to drug release rates independent of the hydrogel mesh size.
Compliant (soft) hydrogel system accessible, due to reliance on vesicle stability and not mesh size for drug release kinetics, produces hydrogels well suited for use in mechanically compliant tissues.
Hydrogel mechanics can be modulated via extent of crosslinking partners conjugated to surface of the vesicles.
Multidrug release, with user-defined release kinetics, possible by using different drug-loaded DENs in the same gel.

Embodiments of this invention can be varied as follows:
The DENs can be stabilized through the use of cleavable dithiol linkages that rapidly degrade under specific conditions (e.g. high pH and/or infrared irradiation). This will result in stabilized DENs within the gel that can undergo a triggered release of the payload when subjected to the appropriate conditions. This provides additional tunability and control over the drug release characteristics of the system.
The physical properties of the hydrogel system can be tuned by modifying the characteristics of the protein-vesicle binding interaction. Functionalization of the vesicles with peptides with more repeats of the P1 peptide motif (i.e. a P2, or P3, etc.) would result in greater binding affinity and a greater hydrogel modulus, while maintaining injectability of the system. Similarly, changes to the C7 protein to tune the binding kinetics will alter the mechanical properties of the gel.
These gels can be made using any sort of complementary binding system, including, but not limited to, host-guest chemistry, DNA complementary strands, and dynamic covalent crosslinking by changing the surface functionalization of the DENs and the binding domain of the biopolymer used in the hydrogel system.
Alternative biocompatible polymers can be used in the place of C7. Biopolymers such as hyaluronic acid, alginate, collagen, PEG, and others can be modified to serve as the complementary component to surface functionalized DENs.

Embodiments of the invention could be of interest to biomedical companies developing materials for drug delivery. Protection and tunable release of growth factors, antibodies, oligonucleotides, and small molecules is key to improving many regenerative and healing treatments. Also, embodiments of the invention could be of interest for biomedical companies developing stem cell transplant therapies. Co-delivery of cells with drug depots providing controlled release of key growth and signaling factors can lead to improved efficacy of treatment in an all-in-one package, and alleviate much of the need for repeat interventions or additional drug treatments. The co-delivery of cells with pro-survival growth factors could provide a more hospitable environment upon delivery into otherwise harsh damaged tissues. Also, embodiments of the invention could be of interest for biotechnology companies developing materials as bioinks for 3D bioprinting. The use of drug-loaded DENs within a bioink would result in 3D printed structures with spatial patterning of releasable drugs such as growth factors.

Embodiments of the invention, like the C7-DEN hydrogel drug delivery vehicle, has advantages over conventional drug delivery hydrogels and the hetero-assembled MITCH-PEG hydrogel system, for example:
The C7-DEN is always injectable due to the reversible crosslinks used for gelation. This allows for the hydrogel to be injected at any point after mixing of the components without any concern for curing times, or loss of mechanical properties.
C7-DEN hydrogels provide independent control of hydrogel mechanics and drug release rates, which are otherwise coupled to the hydrogel mesh size. Here, tuning drug release is achieved by altering the stability of the vesicles and not the mesh size of the hydrogels. This allows the fabrication of hydrogels capable of multiple release rates by using DENs tuned to varied levels of chemical stabilization, while maintaining compliant mechanics necessary for application in mechanically sensitive tissue environments (e.g. within the brain or heart myocardium).
Embodiments also hold advantages over drug releasing vesicles that are not incorporated within a hydrogel by effectively localizing and immobilizing the drug delivery to a specified injection site. Unincorporated vesicles would result in loss of drug release efficacy due to vesicles drifting from the application site, and potentially leading to an increase of adverse side effects associated with non-localized drug release.
Embodiments also hold advantages over drug releasing vesicles that are not incorporated within a hydrogel because the hydrogel viscoelasticity effectively provides mechanical protection to the vesicles during the injection procedure. Unincorporated vesicles are often broken into fragments during the injection procedure, resulting in loss of drug encapsulation, and potentially leading to an increase of adverse side effects associated with bolus drug delivery.
The hydrogel drug delivery vehicle of this invention is also cell compatible, thus allowing for co-delivery of cells and drugs within the same injection. This provides opportunities for the design and application of advanced cell therapies and regenerative medicine strategies that require both cellular and pharmaceutical components.

### Protocol for Drug Loaded DENs Hydrogels

In one exemplary embodiment of a protocol one can consider taking dried MPB and DOPC (at a 1:1 stoichiometric molar ratio) and resuspend with a neutral pH buffered aqueous solution (such as pH 7.4 phosphate buffered saline) containing the payload.

The aqueous lipids-payload mixture is agitated with repeated sonication (for 5 mins intervals) and vortexing (for 1 min intervals) over a 30 min to 1 hr period.

Calcium chloride is added following agitation to result in a final solution of 1-2 μmol lipids/mL, and a 10 mM calcium chloride solution.

A dithiol (such as dithiothreitol) is added in a solution of neutral pH buffered solution at sub-stoichiometric molar ratio to the MPB in the solution to provide chemically stabilization and allowed to react at 37° C. for 30 min-1 hr.

A thiol-functionalized tether group (from 0.1 to 1 molar ratio to the original MPB) is added in a neutral pH buffered solution to surface functionalize the vesicles and allowed to react at 37° C. for 30 min−1 hr.

The resulting chemically-stabilized and surface-functionalized vesicles are washed and recovered using centrifugations to pellet the vesicles followed by removal of the supernatant and resuspension of the vesicle pellet with a buffered solution. The resulting vesicles can be resuspended in 3% sucrose solution and freeze dried for long-term storage if not intended to be used immediately following fabrication.

Formation of the hydrogel is done by mixing a C7 protein solution in a neutral pH with a solution of DENs, with a final concentration of 5-20% C7 (mass per volume) and 0.1-1% DENs (dried DENs mass per volume).

### Sequences

For further and specific teachings of the MITCH system, including the teachings of the first component (C7) as part of the injectable hydrogel in this invention, the teachings of U.S. Pat. No. 9,011,914 as well as the included sequence listings are hereby incorporated to this application by reference for all that it teaches. Relevant Sequence Listings for the purposes of this invention are:

SEQ ID 1:
Arg Leu Pro Ala Gly Trp Glu Gln Arg Met Asp Val Lys Gly Arg Pro Tyr Phe Val
Asp His Val Thr Lys Ser Thr Thr Trp Glu Asp Pro Arg Pro Glu
SEQ ID 2:
Pro Leu Pro Pro Gly Trp Glu Glu Arg Thr His Thr Asp Gly Arg Val Phe Phe Ile Asn
His Asn Ile Lys Lys Thr Gln Trp Glu Asp Pro Arg Met Gln
SEQ ID 3:
Glu Tyr Pro Pro Tyr Pro Pro Pro Pro Tyr Pro Ser Gly
SEQ ID 4:
Ala Gly Ala Gly Ala Gly Pro Glu Gly Ala Gly Ala Gly Ala Gly Pro Glu Gly Arg Gly
Asp Ser Ala Gly Pro Glu Gly Ala Gly Ala Gly Ala Gly Pro Glu Gly Ala Gly Ala Gly Ala Gly Pro
Glu Gly
SEQ ID 5:
Met Gly Ser Ser His His His His His His Ser Ser Gly Leu Val Pro Arg Gly Ser Ser
Ser Gly His Ile Asp Asp Asp Asp Lys Val Asp Gly Thr
SEQ ID 6:
Met Gly Ser Ser His His His His His His Ser Ser Gly Leu Val Pro Arg Gly Ser Ser
Ser Gly His Ile Asp Asp Asp Asp Lys Val Asp Gly Thr Arg Leu Pro Ala Gly Trp Glu Gln Arg
Met Asp Val Lys Gly Arg Pro Tyr Phe Val Asp His Val Thr Lys Ser Thr Thr Trp Glu Asp Pro
Arg Pro Glu Gly Thr Leu Asp Glu Leu Ala Gly Ala Gly Ala Gly Pro Glu Gly Ala Gly Ala Gly
Ala Gly Pro Glu Gly Arg Gly Asp Ser Ala Gly Pro Glu Gly Ala Gly Ala Gly Ala Gly Pro Glu
Gly Ala Gly Ala Gly Ala Gly Pro Glu Gly Glu Leu Leu Asp Gly Thr Arg Leu Pro Ala Gly Trp
Glu Gln Arg Met Asp Val Lys Gly Arg Pro Tyr Phe Val Asp His Val Thr Lys Ser Thr Thr Trp
Glu Asp Pro Arg Pro Glu Gly Thr Leu Asp Glu Leu Ala Gly Ala Gly Ala Gly Pro Glu Gly Ala
Gly Ala Gly Ala Gly Pro Glu Gly Arg Gly Asp Ser Ala Gly Pro Glu Gly Ala Gly Ala Gly Ala
Gly Pro Glu Gly Ala Gly Ala Gly Ala Gly Pro Glu Gly Glu Leu Leu Asp Gly Thr Arg Leu Pro
Ala Gly Trp Glu Gln Arg Met Asp Val Lys Gly Arg Pro Tyr Phe Val Asp His Val Thr Lys Ser
Thr Thr Trp Glu Asp Pro Arg Pro Glu Gly Thr Leu Glu
SEQ ID 7:
Met Gly Ser Ser His His His His His His Ser Ser Gly Leu Val Pro Arg Gly Ser Ser
Ser Gly His Ile Asp Asp Asp Asp Lys Val Asp Gly Thr Pro Leu Pro Pro Gly Trp Glu Glu Arg
Thr His Thr Asp Gly Arg Val Phe Phe Ile Asn His Asn Ile Lys Lys Thr Gln Trp Glu Asp Pro Arg
Met Gln Gly Thr Leu Asp Glu Leu Ala Gly Ala Gly Ala Gly Pro Glu Gly Ala Gly Ala Gly Ala
Gly Pro Glu Gly Arg Gly Asp Ser Ala Gly Pro Glu Gly Ala Gly Ala Gly Ala Gly Pro Glu Gly
Ala Gly Ala Gly Ala Gly Pro Glu Gly Glu Leu Leu Asp Gly Thr Pro Leu Pro Pro Gly Trp Glu
Glu Arg Thr His Thr Asp Gly Arg Val Phe Phe Ile Asn His Asn Ile Lys Lys Thr Gln Glu Asp Pro
Arg Met Gln Gly Thr Leu Asp Glu Leu Ala Gly Ala Gly Ala Gly Pro Glu Gly Ala Gly Ala Gly
Ala Gly Pro Glu Gly Arg Gly Asp Ser Ala Gly Pro Glu Gly Ala Gly Ala Gly Ala Gly Pro Glu
Gly Ala Gly Ala Gly Ala Gly Pro Glu Gly Glu Leu Leu Asp Gly Thr Pro Leu Pro Pro Gly Trp
Glu Glu Arg Thr His Thr Asp Gly Arg Val Phe Phe Ile Asn His Asn Ile Lys Lys Thr Gln Trp Glu
Asp Pro Pro Arg Met Gln Gly Thr Leu Glu
SEQ ID 8:
Ala Gly Ala Gly Ala Gly Pro Glu Gly Ala Gly Ala Gly Ala Gly Pro Glu Gly

### Cell-Adhesive Peptides

In an embodiment of the present disclosure, a cell-adhesive peptide can be associated with (attached directly or indirectly) the viscoelastic hydrogel or with the first protein and/or the second protein prior to forming the viscoelastic hydrogel. In an embodiment, the cell-adhesive peptide is incorporated into the first component, the C7 protein. Table 1 includes a listing of some exemplary cell-adhesive peptides and their putative receptors.

TABLE 1
Examples of cell-adhesive peptides for inclusion in the hydrogels. List modified from
Table I in “Functional peptide sequences derived from the extracellular matrix glycoproteins
and their receptors: Strategies to improved neuronal regeneration” by Sally Meiners,
Mary Lynn T. Mercado and published in Molecular Neurobiology 2003-27(2): 177-195.

| Peptide | Molecular Origin | Domain or Region | Putative Receptor |
|---|---|---|---|
| VFDNFVLK | Tenascin-C | fnD | α7β1 integrin |
| EIDGIELT | | fzn3 | α9β1 integrin |
| RGD | | fn3 | α8β1, αvβ3 integrins |
| RGD | Fibronectin | fn10 | α3β1, α5β1, α7β1, α8β1, αvβ1, |
| | | | αvβ3, αvβ6 integrins |
| LDV | | IIICS-CS I | α4β1 integrin |
| REDV | | IIICS-CS V | α4β1 integrin |
| EDGIHEL | | EDA | α4β1, α9β1 integrins |
| RDIAEIIKDI | Laminin-1 | γ 1 chain | Unidentified G-protein coupled receptor |
| IKVAV | | α 1 chain | β-amyloid precursor protein (APP) |
| YIGSR | | β 1 chain | 67 kDa YIGSR-binding protein |
| IKLLI | | α 1 chain | α3β1 integrin |
| RGD | | α 1 chain | α6β1, αvβ3 integrins |
| RKRLQVQLSIRT | | α 1 chain | |
| KNRLTIELEVRT | Laminin-2 | α 1 chain | |
| LRE | s-Laminin | α 2 chain | |

What is claimed is:

1. An injectable hydrogel for drug delivery, comprising:
(a) a first component of repeating protein domains; and
(b) a second component cross-linked with the first component, wherein the second component is an inter-bilayer crosslinked multilamellar vesicle, wherein the vesicle is capable of containing drug cargo and eluting the drug cargo, wherein the surface of the vesicle has tethers, wherein protein domains of the first component are cross-linked with the tethers.

2. The injectable hydrogel as set forth in claim 1, wherein the tethers are proline peptide tethers.

3. The injectable hydrogel as set forth in claim 1, wherein the vesicle is derived from a first lipid and a second lipid.

4. The injectable hydrogel as set forth in claim 3, wherein the first lipid is 1,2-dioleoyl-sn-glycero-3-phosphocholine (DOPC).

5. The injectable hydrogel as set forth in claim 3, wherein the second lipid is 1,2-dioleoyl-sn-glycero-3-phosphoethanolamine-N-[4-(p-maleimidophenyl) butyramide] (MPB).

6. The injectable hydrogel as set forth in claim 1, wherein the protein domain in the repeating protein domains is a WW protein sequence interspersed with hydrophilic protein sequences, wherein the WW protein sequence is selected from the group consisting of SEQ ID 1, SEQ ID 2, SEQ ID 3, SEQ ID 4, SEQ ID 5, SEQ ID 6, and SEQ ID 7.

7. The injectable hydrogel as set forth in claim 1, wherein the repeating ranges from 2 to 10.
